(19) United States
(12) Plant Patent Application Publication
(10) Pub. No.: US 2003/0056267 P1
(43) Pub. Date: Mar. 20, 2003

(54) Chrysanthemum plant named 'Day Dream Yellow'

(76) Inventor: Mark Roland Boeder, J H Den Haag (NL)

Correspondence Address: HOWREY SIMON ARNOLD & WHITE, LLP, BOX 34, 301 RAVENSWOOD AVE., MENLO PARK, CA, 94025, US

(21) Appl. No.: 09/957,488

(22) Filed: Sep. 19, 2001

(51) Int. Cl. A01H 5/00

(52) U.S. Cl. PLT/295

(57) ABSTRACT

A new variety of Chrysanthemum plant named ‘DAY DREAM YELLOW,’ having a medium vigour of branching habit with many yellow anemone centred medium yellow flowers with a darker yellow green cushion.

### BACKGROUND OF THE INVENTION

The present invention comprises a new and distinct cultivar of Chrysanthemum botanically known as Dendranthema grandiflora, and referred to by the cultivar name ‘Day Dream Yellow’.

‘Day Dream Yellow’, identified as 20164-4 originated from an irradiated mutation of a whole plant of ‘Dream Time’ in Chichester, West Sussex, United Kingdom. The new variety ‘Day Dream Yellow’ has been asexually reproduced by vegetative cuttings at Chichester, West Sussex, United Kingdom and the distinguishing characteristics are retained through successive generations of asexual reproduction.

### BRIEF SUMMARY OF THE INVENTION

‘Day Dream Yellow’ is a pot type of Chrysanthemum plant variety having a flat capitulum of yellow anemone type flowers.

### Comparison with Parent

Plants of the new Chrysanthemum variety ‘Day Dream Yellow’ are similar to the parent cultivar ‘Dream Time’ in plant habitat and growth rate. In side-by-side comparisons in Chichester, West Sussex, United Kingdom, under commercial practice, plants of the new Chrysanthemum variety ‘Day Dream Yellow’ differed from plants of the parent cultivar ‘Dream Time’ in the following characteristics.

1. The new variety ‘Day Dream Yellow’ produces yellow anemone flowers, whereas the parent cultivar ‘Dream Time’ produces white anemone flowers.

2. Plants of the new variety ‘Day Dream Yellow’ have similar inflorescence than plants of the parent cultivar ‘Dream Time.’

3. ‘Day Dream Yellow’ has disc florets that are more yellow than the parent ‘Dream Time.’

4. ‘Day Dream Yellow’ has a response time that is four days faster than the parent ‘Dream Time.’

### Comparison with Other Varieties

Plants of the new Chrysanthemum variety ‘Day Dream Yellow’ are similar to the cultivar ‘Yellow Merced’ in plant habitat and growth rate. However, in side-by-side comparisons in Chichester, West Sussex, United Kingdom, under commercial practice, plants of the new Chrysanthemum variety ‘Day Dream Yellow’ differed from plants of the cultivar ‘Yellow Merced’ in the following characteristics.

1. The new variety ‘Day Dream Yellow’ produces larger style flowers, whereas the cultivar ‘Yellow Merced’ produces smaller style flowers.

2. Plants of the new variety ‘Day Dream Yellow’ have larger and taller inflorescence than plants of the parent cultivar ‘Yellow Merced.’

3. The new variety ‘Day Dream Yellow’ had a response time that was two days slower than ‘Yellow Merced.’

4. ‘Day Dream Yellow’ produces plants that are shorter in vigour and more yellow in colour than ‘Yellow Merced.’

### BRIEF DESCRIPTION OF ILLUSTRATIONS

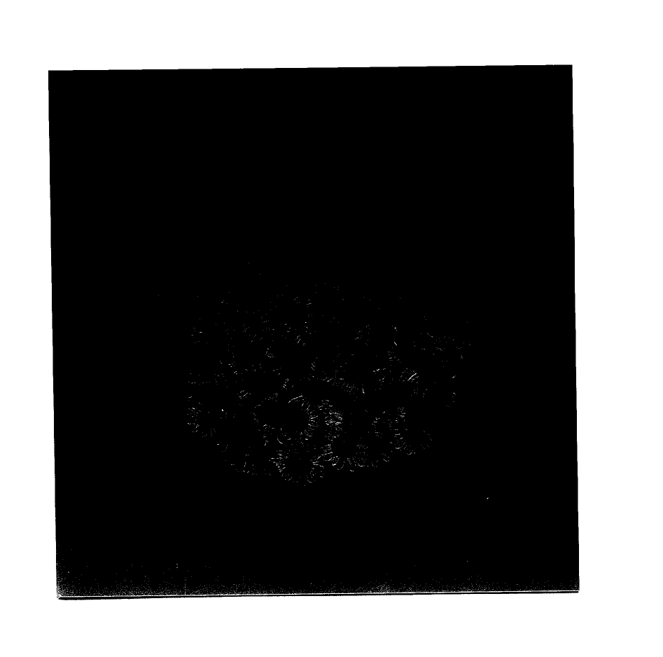

Typical specimens of the plant and flowers for the new Chrysanthemum variety ‘Day Dream Yellow’ are shown in the accompanying photograph. The colours shown are as true as possible within the usual limits of this kind of illustration. FIG. 1 is a whole plant view of the new variety ‘Day Dream Yellow’ grown in a pot. The plant shown in the illustration is 56 days from the start of Short Days.

### DETAILED BOTANICAL DESCRIPTION

The following description of the new Chrysanthemum variety ‘Day Dream Yellow’ is of plants grown in a greenhouse in Chichester, West Sussex, United Kingdom in the month of August. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 24° C. to 25° C. during the day under light conditions of about 5,000 to 6,000 foot candles. The plants respond well to the use of growth retardant, such as one B9 treatment at about 2000 ppm. To produce a commercial product the plants may be pinched once with the centre bud removed. The typical container size for commercial growth is 14 cm. It has been observed that the shelf life of the new variety is about 22 days with a response time of about 7½ weeks. The new variety is suitable for growth in a temperature range of 16° C. to 25° C.

The new variety may be produced as a Spray. The following description is with respect to a plant produced as a pot Chrysanthemum. In the description of this new Chrysanthemum variety, colour values have been taken from The Royal Horticultural Society Colour Chart (R.H.S.C.C.).

### Plant

Plant type: Pot

Habit: Upright and branching

Height: Short/Medium 16 to 19 cm

Width: Near 28 cm

Branching characteristics: Spreading

Length of Lateral Branches: 6 to 10 cm

Number of Breaks from Pinch: 4 to 5

Stem Colour: 146 A

Response Time: 51 days

Vigour: Medium

Shelf Life: Near 22 days

Disease: No observations

Pest: No Observations

Growth retardant type and treatment: B9 85% 1 application of 2 gram/litre at 21 days after propagating unrooted vegetative cuttings. The plants were grown for two weeks in Long Day conditions (20 hours of light) and then transferred to Short Day conditions (13 hours of dark).

Propagation:

Type.—Vegetative propagation via stem cuttings.

Time to rooting.—12-14 days with soil temperatures of 18° C.

Rooting habit.—Fine and fibrous.

### Foliage

Number of Leaves per Lateral Branch: 5 to 9

Compound or single: Single

Arrangement of Leaves: Alternate

Shape of leaf.—Typically 5 to 6 lobed, oblong ovate.

Size of leaf.—Width (cm): Near 3.5 cm Length (cm): Near 5.5 cm.

Leaf apex.—Rounded.

Base.—Obtuse.

Attachment.—Petioled.

Aspect.—Slightly undulating.

Margin.—Palmately lobed.

Surface characteristics.—Top: Slightly pubescent. Bottom: Slightly pubescent

Petiole: Colour: Near 137 B Length: Near 0.8 cm

Venation: Net, prominent midvein at under side.

Colour.—upper side: Near 138 B. underside: Near 138 B

Colour: Mature Leaf, upper side: Near 137 A; under side: Near 137 C Young Leaf, upper side: Near 137 B; under side: Near 137 C

### Flower

Flower Appearance: Flat and circular

Flower type: Anemone

Flower Form: Flat

Flower shape: Circular

Flower Habit: Upright

Number of blossoms per branch: 3 to 5

Inflorescence form: Cyme

Depth of fully expanded blossoms.—Near 1.0 cm.

Diameter of fully expanded blossoms.—Near 2.5 to 3 cm.

Anemone centre diameter.—Near 2 cm.

Sepals:

Number.—18 to 20.

Colour.—Near 137 B.

Length.—0.8 to 1.0 cm.

Width.—Near 0.2 cm.

Texture/Appearance.—Slightly pubescent.

Peduncles: Peduncle length is shortest at the terminal flower and progressively longer moving down the stem; longest length is on average 5 cm; shortest length on average 2 cm.

Length.—3 cm.

Colour.—Near 137 B.

Surface.—Slightly pubescent.

Habit.—Slightly curving.

Strength.—Medium.

Pedicel:

Length.—Terminal: Near 3.5 cm; Lateral: Near 5 cm.

Colour.—Near 137 B.

Surface.—Slightly pubescent.

Habit.—Slightly curving.

Strength.—Medium.

Ray Florets:

Form/Shape.—Straight, rounded edge.

Texture/Appearance.—Matte.

Number per flower.—24 to 35.

Length.—Near 1.0 cm; Width: Near 0.3 to 0.5 cm.

Apex.—Rounded.

Base.—Tapered.

Margin.—Entire.

Disc florets:

Form/Shape.—Petaloid.

Texture/Appearance.—Shiny.

Number per flower.—Approx. 50.

Length.—Near 0.5 cm; Width 0.01 cm.

Diameter of disc.—Near 1.1 cm.

Fragrance: Little

Flower Bud (at onset of colour):

Length.—Near 0.5 cm.

Diameter.—Near 1.0 cm.

Form/shape.—Globular.

General flower colour: Yellow

1. Ray florets, upper side.—Immature: Near 6 A Mature: Near 6 B Older/Fading: Near 6 C.

2. Ray florets, under side.—Immature: Near 6 B Mature: Near 6 C Older/Fading: Near 6 D.

3. Disc florets.—Immature: Near 150 A Mature: Near 150 B Older/Fading: Near 150 C.

4. Bud.—Near 137 A.

Flower progression: Slight colour fading during ageing.

### Reproductive Organs

Ray Florets: Pistillate

Pistil number.—35 to 40.

Stigma colour.—Near 6 C.

Stigma shape.—Forked.

Style colour.—Near 6 C.

Style length.—Near 0.5 cm.

Disc Florets: Pistillate

Pistil number.—<100.

Stigma colour.—Near 6 C.

Stigma shape.—Forked.

Style colour.—Near 6 C.

Style length.—Near 0.5 cm.

Staminate.—Not Observed

What is claimed is:

1. A new and distinct variety of Chrysanthemum plant, substantially as described and illustrated herein.
